(19) United States
(12) Patent Application Publication
(10) Pub. No.: US 2013/0150330 A1
(43) Pub. Date: Jun. 13, 2013

(54) DECOQUINATE PRODRUGS

(71) Applicants: Stefano A. Pogany, Lawrence, KS (US); Mehmet Tanol, Lawrence, KS (US); Michael J. Baltezor, Lees Summit, MO (US)

(72) Inventors: Stefano A. Pogany, Lawrence, KS (US); Mehmet Tanol, Lawrence, KS (US); Michael J. Baltezor, Lees Summit, MO (US)

(73) Assignee: UNIVERSITY OF KANSAS, Lawrence, KS (US)

(21) Appl. No.: 13/495,772

(22) Filed: Jun. 13, 2012

Related U.S. Application Data
(60) Provisional application No. 61/496,372, filed on Jun. 13, 2011.

(51) Int. Cl. A61K 31/675; A61K 45/06; C07F 9/60

(52) U.S. Cl. CPC A61K 31/675; C07F 9/60; A61K 45/06
USPC 514/82; 546/23

(57) ABSTRACT

A compound can include a structure having decoquinate coupled to a prodrug moiety, or derivative or isomer or pharmaceutically acceptable salt thereof. The compound can be a decoquinate prodrug. The decoquinate prodrug can have a structure of any of the formulae described herein. The decoquinate prodrug can be synthesized in any manner, such as a synthetic method that includes Scheme 1A or Scheme 1B and Schemes 2, 3, and/or 4. The decoquinate prodrug can be prepared into a pharmaceutical composition with a pharmaceutically acceptable carrier, such as an aqueous composition. The decoquinate prodrug can be used for inhibiting or treating a parasitic infection, such as a malarial infection or a coccidian infection.

### CROSS-REFERENCE TO RELATED APPLICATION

This patent application claims the benefit of U.S. Provisional Application Ser. No. 61/496,372, filed on Jun. 13, 2011, which provisional application is incorporated herein by specific reference in its entirety.

### BACKGROUND

Decoquinate (i.e., ethyl 6-(decyloxy)-7-ethoxy-4-oxo-1,4-dihydroquinoline-3-carboxylate or 6-decoxy-7-ethoxy-4-oxo-1H-quinolone-3-carboxylic acid ethyl ester) has been used for veterinary treatments as a coccidiostat. A coccidiostat is an antiprotozoal agent that acts upon coccidia parasites. Coccidia are a subclass of microscopic, spore-forming, single-celled obligate parasites belonging to the apicomplexan class Conoidasida. Coccidian parasites can infect the intestinal tracts of animals, and are the largest group of apicomplexan protozoa. Coccidia are obligate, intracellular parasites that live and reproduce within an animal cell. These microorganisms form a subclass within the Conoidasida, and are divided into four orders distinguished by the presence or absence of various asexual and sexual stages.

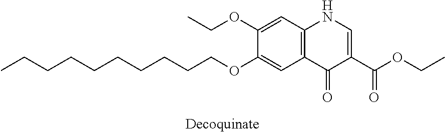 [SMILES: CCCCCCCCCCOc1cc2c(=O)c(C(=O)OCC)c[nH]c2cc1OCC]

### DESCRIPTION OF FIGURES

FIG. 1A shows a reaction for Scheme 1A.

FIG. 1B shows a reaction for Scheme 1B.

FIG. 2 shows a reaction for Scheme 2.

FIG. 3 shows a reaction for Scheme 3 and a reaction for Scheme 4.

### DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention includes a water-soluble prodrug of decoquinate, such as a phosphoryloxyalkyl decoquinate (decoquinate-POA) or phosphoryloxymethyl decoquinate (decoquinate-POM). As such, the prodrug includes a modified decoquinate molecule having a prodrug moiety. The decoquinate prodrug can have an increased solubility in water (>50 mg/mL), whereas decoquinate itself is poorly soluble in water (<0.05 mg/ml). Improved solubility in water allows administration of the decoquinate prodrug by the intravenous and intramuscular routes. Additionally, water solubility also makes oral administration feasible by improving absorption from the gastrointestinal tract (GI tract).

The POA can be as shown below as having the oxygen atoms covalently bonded to substituents (e.g., POA¹) or ionically associated with cationic counterions (e.g., POA²). The POM includes a hydrogen for each of R¹ and R².

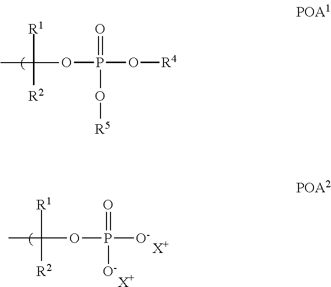

Malaria is an infectious disease caused by eukaryotic protists that act as parasites in red blood cells, causing symptoms of fever and headache which can lead to coma or death. There are a number of these parasites that can cause malaria, which can include P. falciparum, P. vivax, P. ovale, P. malariae, and P. knowlesi. The pathogens that cause malaria are from a different class than coccidia parasites, and also have different pathogenic pathways. As such, a treatment for a coccidia parasite that infects the GI tract would not be suspected of treating a malaria infection of the blood or red blood cells. Thus, it would be surprising and unexpected if an agent for treating coccidia parasites in the GI tract would be useful for treating malaria parasites in the red blood cells.

Now, the decoquinate prodrugs described herein have been found as useful in a potential treatment of malaria. Accordingly, the decoquuiinate prodrugs described herein can be administered to a suject in need thereof in order to inhibit or treat a malaria infection. Information regarding use of the decoquinate prodrugs in prevention, inhibition, or treatment of malaria can be found in: Cruz et al. “Drug Screen Targeted at Plasmodium Liver Stages Identifies a Potent Multistage Antimalarial Drug,” Journal of Infectous Disease; 205:1278-86 (2012), which is incorporated herein by specific refernece in its entirety.

In one embodiment, the present invention can include a prodrug of decoquinate or derivative or salt thereof. The decoquinate prodrug can include a prodrug entity that releases decoquinate when acted upon by phosphoesterase enzymes which are ubiquitous in varrious animals, such as mammals including dogs, cats and humans. Accordingly, the decoquinate prodrug can include a decoquinate structure having a prodrug entity covalently coupled thereto. The prodrug moiety can be coupled to any atom of the decoquinate molecule. Also, the derivative of the decoquinate prodrug can include any suitable derivative of decoquinate that has biological activity as described herein, where the decoquinate can have any of the common chemical moiety substituents known in the art, such as those described herein. The salt of the decoquinate prodrug can be any suitable salt, such as a pharmaceutically acceptable salt known in the art.

In one embodiment, the decoquinate prodrug can include the prodrug moiety conjugated to the nitrogen of decoquinate. While other conjugation positions may be suitable, it has been found that covalently linking a prodrug moiety to the nitrogen of decoquinate can produce a prodrug that is capable of being processed or otherwise reacted in a biological system so as to produce free decoquinate or derivative thereof that is biologically active. Accordingly, the decoquinate prodrug can include a structure of Formula 1 or derivative or isomer or pharmaceutically acceptable salt thereof. Here in Formula 1, “Prodrug” is any prodrug moiety, preferably a prodrug moiety that has high water solubility, such as a prodrug moiety that can increase the water solubility of decoquinate. Additionally, the decoquinate prodrug can include a structure of Formula 1A or Formula 1B or Formula 1C or Formula 1D or Formula 1E or derivative or isomer or pharmaceutically acceptable salt thereof, which includes the prodrug moiety that improves water solubility linked to the decoquinate via a linker. The linker can be any type of linker, such as the linkers described herein. Also, the linker can be an alkyl linker with “n” being any integer, such as from 0 or 1 to 20, 1 to 15, 1 to 10, 1 to 5, or the like. When “n” is 0, the POA or POM is linked directly to the nitrogen.

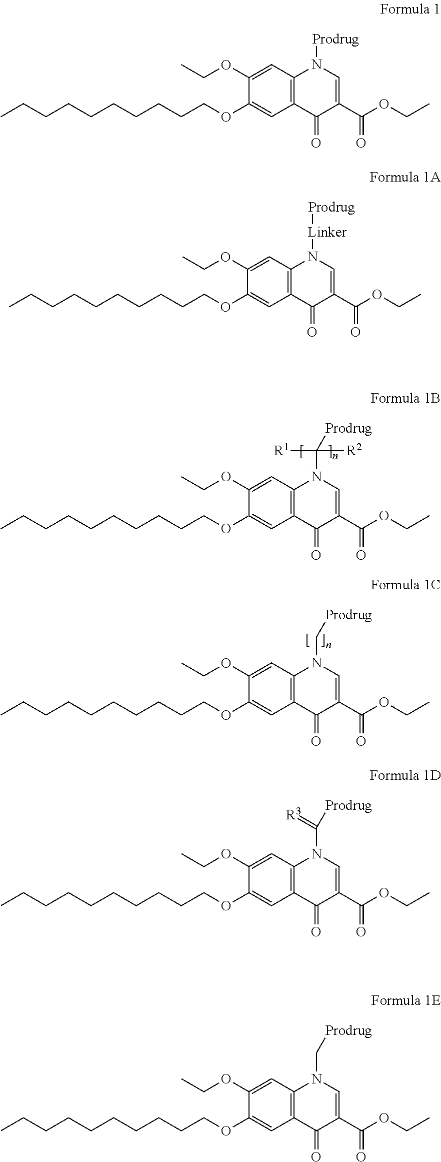

In one embodiment, the decoquinate prodrug can include a prodrug moiety that includes a phosphoryloxyalkyl (POA) moiety or salt thereof. The POA moiety salt can be any pharmaceutically acceptable salt. The prodrug moiety may also include a linker coupled to a phoshoryloxy (PO) group, which is shown below as having the oxygen atoms covalently bonded to substituents (e.g., PO¹) or ionically associated with cationic counterions (e.g., PO²). The PO group can also be linked to decoquinate through various linker entities as shown in the formulae and described herein. Accordingly, the decoquinate prodrug can include a structure of Formula 2 or derivative or isomer or pharmaceutically acceptable salt thereof. Additionally, the decoquinate prodrug can include a structure of Formula 2A or Formula 2B or Formula 2C or Formula 2D or Formula 2E or derivative or isomer or pharmaceutically acceptable salt thereof, which includes the PO prodrug moiety that improves water solubility linked to the decoquinate via a linker. Also, the linker can be an alkyl linker with “n” being any integer, such as from 1 to 20, 1 to 15, 1 to 10, 1 to 5, or the like.

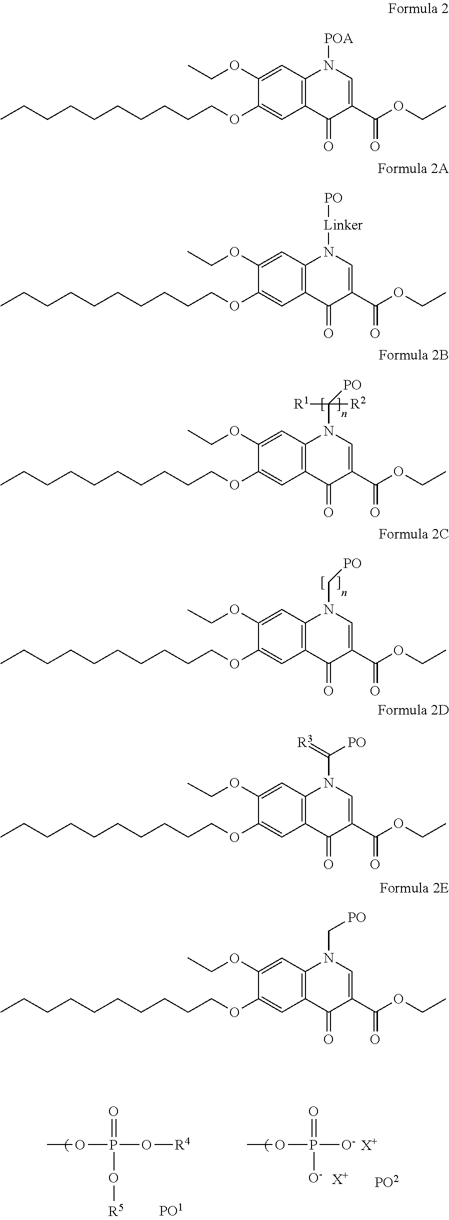

In one embodiment, the decoquinate prodrug can include a prodrug moiety that includes an etherphosphoryloxy (EPO) moiety or salt thereof. The EPO moiety salt can be any pharmaceutically acceptable salt. The prodrug moiety may also include a linker coupled to a etherphoshoryloxy (EPO) group, which is shown below as having the oxygen atoms covalently bonded to substituents (e.g., EPO¹) or ionically associated with cationic counterions (e.g., EPO²). The EPO group can also be linked to decoquinate through various linker entities as shown in the formulae. Accordingly, the decoquinate prodrug can include a structure of Formula 3 or Formula 4 or derivative or isomer or pharmaceutically acceptable salt thereof. Additionally, the decoquinate prodrug can include a structure of Formula 3A or Formula 3B or Formula 3C or Formula 3D or Formula 3E or derivative or isomer or pharmaceutically acceptable salt thereof, which includes the EPO prodrug moiety that improves water solubility linked to the decoquinate via a linker. Additionally, the decoquinate prodrug can include a structure of Formula 4A or Formula 4B or Formula 4C or Formula 4D or Formula 4E or derivative or isomer or pharmaceutically acceptable salt thereof, which includes the EPO prodrug moiety that improves water solubility linked to the decoquinate via a linker. Also, the linker can be an alkyl linker with “n” being any integer, such as from 1 to 20, 1 to 15, 1 to 10, 1 to 5, or the like.

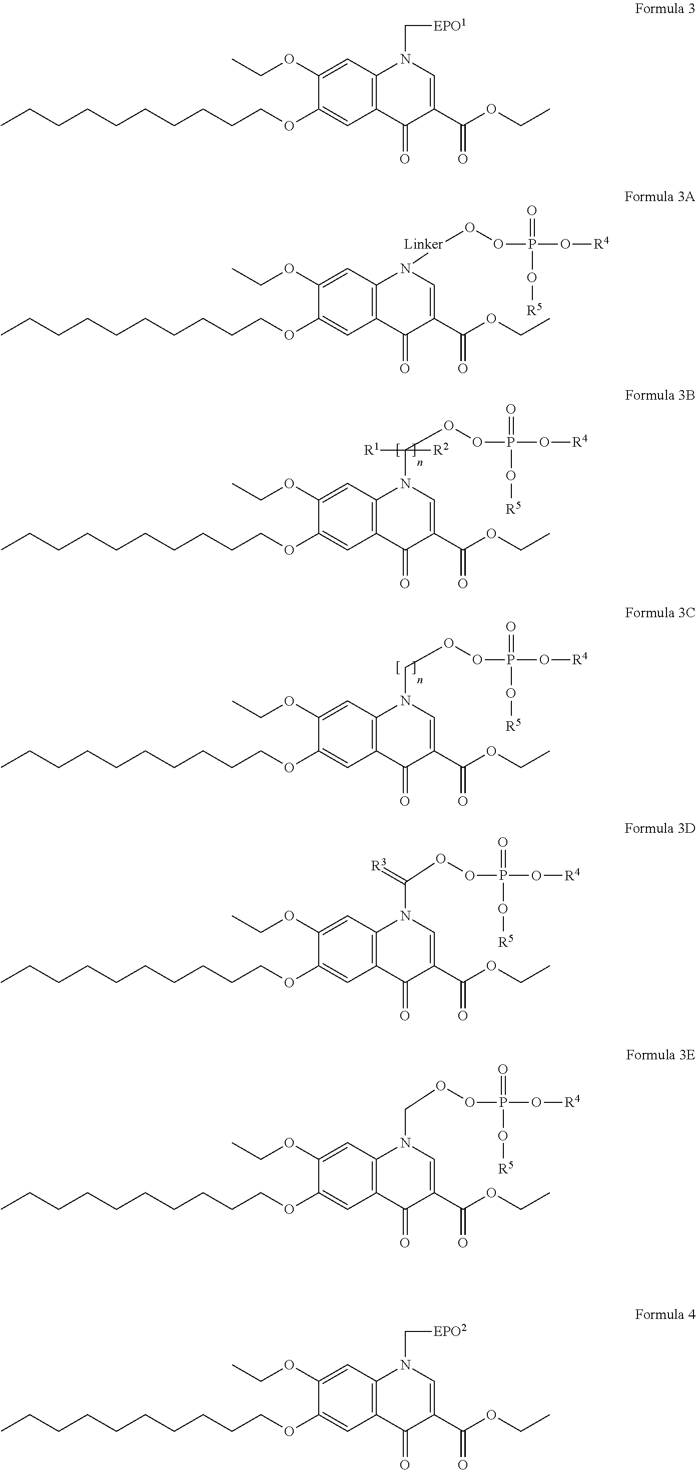

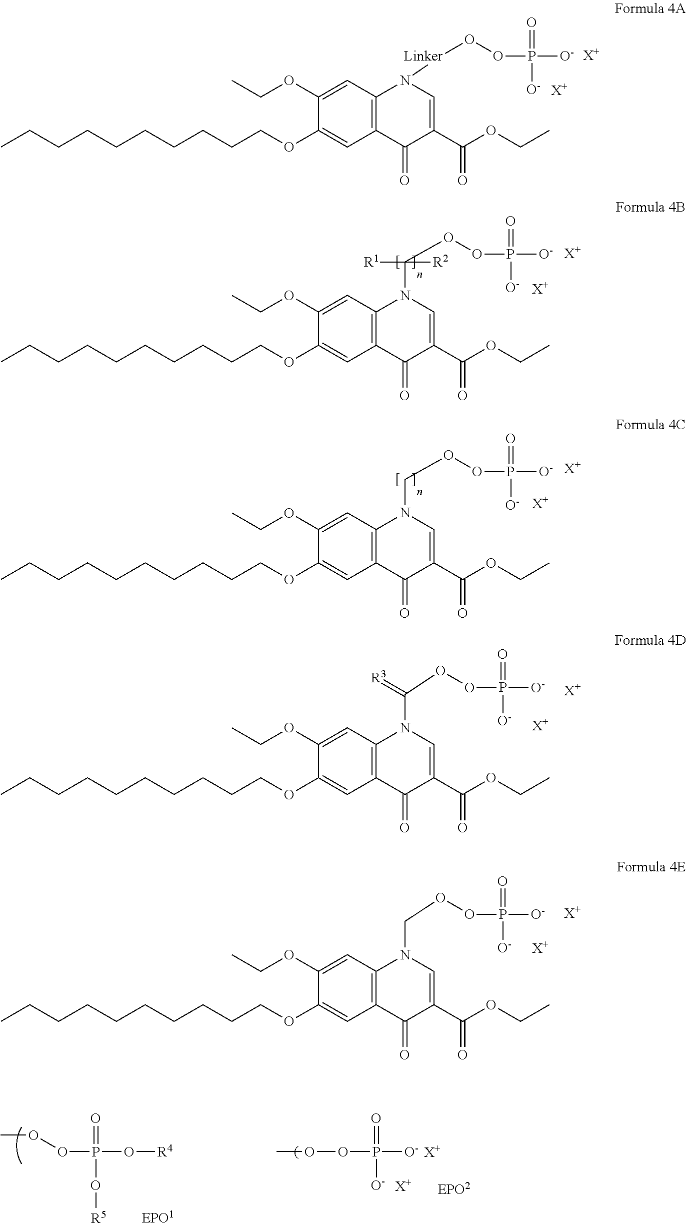

In one embodiment, the R⁴ and/or R⁵ can include one or more of the protecting group substituents tert-butyl or benzyl. This embodiment can be an intermediate or reagent that is prepared into a biologically useful prodrug; however, it is possible that this embodiment can also be used as a prodrug.

In one embodiment, the decoquinate prodrug can include a prodrug moiety that includes phosphoryloxymethyl (POM) moiety or salt thereof. The POM moiety salt can be any pharmaceutically acceptable salt. Accordingly, the decoquinate prodrug can include a structure of Formula 5 or Formula 6 or derivative or isomer or pharmaceutically acceptable salt thereof.

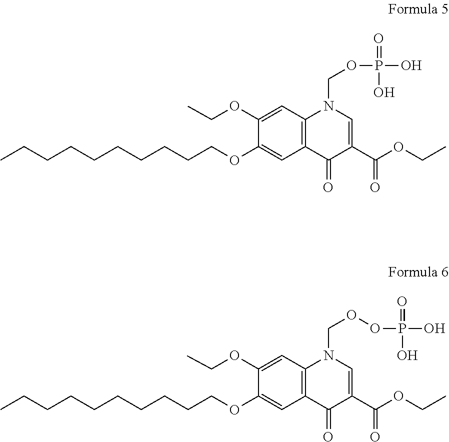

In one embodiment, the substituents R¹, R², R³, R⁴ and R⁵ of any of the formulae can be independently selected from or include hydrogen, halogens, hydroxyls, alkoxys, straight aliphatics, branched aliphatics, cyclic aliphatics, heterocyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, sugars, sugar mimics, derivatives thereof, or combinations thereof as well as other well-known chemical substituents. The aliphatic groups can include carbon chains, each independently being about 1-20, about 1-10, or about 1-5 carbons, which carbons may be substituted with hetero atoms O, N, S, P, or the like.

In one embodiment, the “n” of any of the formula can range from 1 to 20, or from 1 to 15, or from 1 to 10, or from 1 to 5 or be 1 or 2. When the alkyl group of POA or methyl group of POM is considered part of the prodrug moiety, n can be 0.

In one embodiment, the substituents R¹, R², R³, R⁴ and R⁵ of any of the formulae can be independently selected from or include any linear, branched, or cyclic aliphatic group having from C₁ to C₂₀ that is substituted or unsubstituted with a substituent. Examples can include methyl, ethyl, propyl, isopropyl, butyl, tert-butyl, cyclopropane, cyclobutane, cycloheptate, cyclohexane, or combinations thereof or derivatives thereof, whether substituted or unsubstituted, or the like. Also, the aliphatic groups can have one or more backbone carbons substituted with one more hetro atoms.

In one embodiment, the substituents R¹, R², R³, R⁴ and R⁵ of any of the formulae can be independently selected from or include any aromatic group that is monocyclic or polycyclic, such as benzene groups, toluene groups, ethylbenzene groups, p-xylene groups, m-xylene groups, mesitylene groups, durene groups, 2-phenylhexane groups, biphenyl groups, phenol groups, aniline groups, nitrobenzene groups, benzoic acid groups, naptholene groups, acenaphthene, acenapthylene, anthracene, chrysene, fluoranthene, phenathrene, pyrene, coronene, corannulene, tetracene, pentacene, triphenelene, ovalene, or combinations thereof or derivatives thereof, whether substituted or unsubstituted. When substituted, the substituents can be as described herein. The substituents can also include hetro atoms.

In one embodiment, the substituents R¹, R², R³, R⁴ and R⁵ of any of the formulae can be independently selected from or include any amino acid side group so that the amino acid is selected from positively charged amino acids, arginine, histidine, lysine, negatively charged amino acids, aspartic acid, glutamic acid, polar uncharged amino acids, serine, threonine, asparagine, glutamine, cysteine, selenosystein, glycine, proline, hydrophobic amino acids, alanine, valine, isoleucine, methionine, phenylalanine, tyrosine, tryptophan, other amino acids, non-standard amino acids, carnitine, hydroxyproline, selenomethionine, lanthionine, 2-aminoisobutyric acid, dehydroalanine, gamma-aminobutyric acid, ornithine, citrulline, beta alanine, pantothenic acid, or derivatives thereof.

In one embodiment, the linker of any of the formulae can include a straight aliphatic, branched aliphatic, cyclic aliphatic, heterocyclic aliphatic, substituted aliphatic, unsubstituted aliphatic, saturated aliphatic, unsaturated aliphatic, aromatic, polyaromatic, substituted aromatic, hetero-aromatic, amine, primary amine, secondary amine, tertiary amine, aliphatic amine, carbonyl, carboxyl, amide, ester, amino acid, peptide, polypeptide, sugars, sugar mimic, derivatives thereof, or combinations thereof as well as other well-known chemical linkers.

In one embodiment, the substituents R¹ and R² of any of the formulae can combine in order to form a ring that is aliphatic or aromatic with 3, 4, 5, 6, or 7 members in the ring, where the backbone of the ring can include one or more heteroatoms or include only carbons, which ring may be substituted or unsubstituted.

In one embodiment, R³ can be O, N, S, P, or the like. Alternatively, R³ can be the same as defined for R¹. The atom of R³ bonded to the double bond is not saturated as it has the double bond.

In one embodiment, in any of the formulae the substituents R¹ and R² can be the same and can be different from R⁴ and R⁵ that are the same substituent.

In one embodiment, in any of the formulae the substituents R¹ and R² can be hydrogen and R⁴ and R⁵ can be tert-butyl and/or benzyl.

In one embodiment, in any of the formulae the substituents R¹, R², R⁴, and R⁵ can be hydrogen.

Additionally, any of the decoquinate, decoquinate derivatives, or prodrugs described herein and represented by the formulae can have additional R group substituents that are possible with the chemical structures. Any of the R groups, such as but not limited to R¹, R², R³, R⁴ and R⁵, or any R group substituent in place of any hydrogen can be independently selected from substituents selected from the group of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO —), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—C≡N), isocyano (—N+≡C —), cyanato (—O—C≡N), isocyanato (—O—N+≡C—), isothiocyanato (—S—C≡N), azido (—N═N+═N—), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—S₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)2), phosphonato (—P(O)(O—)₂), phosphinato (—P(O)(O—)), phospho (—PO₂), phosphino (—PH₂), derivatives thereof, and combinations thereof.

The X can be any cationic counterion for the anionic oxygen atoms of the phosphate. As such, examples of X can include Li, Na, K, Rb, Cs, and Fr, with Na being preferred. Also, the X can be a cationic salt with a +1 charge. Examples of such salts can include ammonium, diethylamine, ethylenediamine, or the like.

Any of the chemical terms described herein are defined by their standard chemical definitions. Also, the provisional application incorporated herein provides chemical definitions, which definitions are incorporated herein by specific reference.

In one embodiment, the decoquinate prodrug can include a prodrug entity, such as a POA or POM that improves solubility and bioavailability over decoquinate. The POA and POM moieties have chemical features that promote water solubility, and thereby promote bioavailability.

In one embodiment, a composition can include the decoquinate prodrug as described herein and another substance. The other substance can be, for example, another anti-malarial agent. The anti-malarial agent can be a quinine or related agent, chloroquine, amodiaquine, pyrimethamine, proguanil, sulfonamides, mefloquine atovaquone, primaquine, artemisinin, halofantrine, doxycycline, clindamycin, or suitable derivatives, prodrugs, or salts thereof. These prodrugs can include the prodrug moieties as described herein.

In one embodiment, a composition can include the decoquinate prodrug in a pharmaceutically acceptable carrier. The pharmaceutically acceptable carrier can be any pharmaceutically acceptable carrier known or developed. In one aspect, the pharmaceutically acceptable carrier can include an aqueous composition such as water. The water can be purified water that is pharmaceutically acceptable. The water may also be part of an aqueous solution that is injectable and that has suitable tonicity.

In one embodiment, the decoquinate prodrug can be used in a prophylactic regimen or therapeutic regime for the prevention, inhibition, or treatment of a parasitic infection. The parasitic infection can include a malaria infection or a coccidian infection. Accordingly, the composition can include the decoquinate prodrug in a therapeutically effective amount to be effective in the prophylactic or treatment regimen for the parasitic infection. While the therapeutically effective amount can be for a coccidian infection, in one embodiment it is preferred that the decoquinate prodrug is present in a therapeutically effective amount for a prophylactic or therapeutic treatment for malaria. This can include the decoquinate prodrug being present in a therapeutically effective amount to inhibit or kill a malaria-causing parasite, such as P. falciparum, P. vivax, P. ovale, P. malariae, and P. knowlesi (e.g., P. being for Plasmodium).

In one embodiment, the composition having the decoquinate prodrug can be configured to be suitable for administration to a subject. The administration can be via injection or oral administration. Transdermal administration may also be suitable with proper penetration enhancers. Configuring a drug, such as a water soluble decoquinate prodrug, to be suitable for administration is well within the skill of one of ordinary skill in the art with the disclosure provided herein. The subject can be any mammal or aviary. Preferably, the composition is configured for administration to a human subject, however, administration to cats, dogs, or other domesticated animals may be advantageous.

In one embodiment, a composition can include the decoquinate prodrug is present in an amount greater than 0.01% w/w of the composition. For example, the decoquinate prodrug can be included in the composition in a therapeutically effective amount that ranges from about 0.01% to about 99% to about, from about 0.05% to about 75%, or from about 0.1% to about 50% w/w of the composition to be administered. In fact, the high solubility allows for substantially any amount of the decoquinate prodrug to be included in a composition, especially in an aqueous composition.

The solubility of decoquinate prodrug with the POM prodrug moiety in water has been found to be greater than 50 mg/mL. This result was obtained by adding increasing amounts of the decoquinate-POM prodrug to a vial containing 1 mL of deionized water. Comparatively, decoquinate without the prodrug moiety is practically insoluble in water (<0.05 mg/ml). Improved solubility in water allows administration of the decoquinate prodrug by the intravenous and intramuscular routes. Additionally, water solubility also makes oral administration feasible by improving absorption from the GI tract.

The enhanced solubility of the different embodiments of the decoquinate prodrug allows for practically any type of aqueous formulation to be prepared. Also, the solubility allows for pills or other oral compositions to be used as the decoquinate prodrug can become solubilized in the GI tract for adsorption and improved bioavailability.

The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences handbook, which all versions thereof incorporated herein by specific reference. On this basis, the compositions include, albeit not exclusively, solutions of the substances in association with one or more pharmaceutically acceptable vehicles or diluents, and contained in buffered solutions with a suitable pH and iso-osmotic with the physiological fluids.

In one embodiment, the effective amount of decoquinate prodrug is within the range of about 1 to about 200 mg/kg body weight of a subject. In one aspect, the effective amount of decoquinate prodrug is within the range of about 5 to about 50 mg/kg body weight. The decoquinate prodrug can be prepared into an aqueous, gel, or solid dosage form that contains from about 20 mg to about 1000 mg of decoquinate prodrug. In one aspect, the composition can include about 20 mg to about 200 mg of decoquinate prodrug/kg body weight of subject, and can be formulated into a solid oral dosage form, a liquid dosage form, or an injectable dosage.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (e.g., Tween®), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes as well as any polymeric microsphere. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration. Common carriers or excipients can be used for preparing pharmaceutical compositions designed for such routes of administration. Controlled release compositions, such as in depots or microspheres can be used. The composition can be biodegradable, such as polylactic acid and/or polyglycolic acid or others.

In one embodiment, a method of treatment can include identifying a subject with a parasitic infection, and administering a therapeutically effective amount of the decoquinate prodrug to the subject in order to treat the parasitic infection. Preferably, the parasitic infection is malaria. When a subject travels to a geographical location that has a high incidence of malaria, such as a tropical region, the subject can be in need of the decoquinate prodrug as a prophylactic. After exposure to malaria, the subject can be in need of the decoquinate prodrug for inhibition or treatment of a malaria infection.

In one embodiment, a method for preparing the decoquinate prodrug is provided. The method can include any synthetic method that conjugates the prodrug moiety to the decoquinate, such as through the nitrogen atom of decoquinate. The method can include: providing a decoquinate or derivative thereof; activating the nitrogen on the decoquinate or derivative; coupling the activated nitrogen with a protected prodrug precursor; and deprotecting the protected prodrug. The protected prodrug precursor can include the tert-butyl or benzyl protecting groups on oxygen atoms of a phosphate group. The method can further include desalting the decoquinate prodrug that has cation counter ions, which can provide the decoquinate prodrug having hydrogens on the prodrug moiety as described herein.

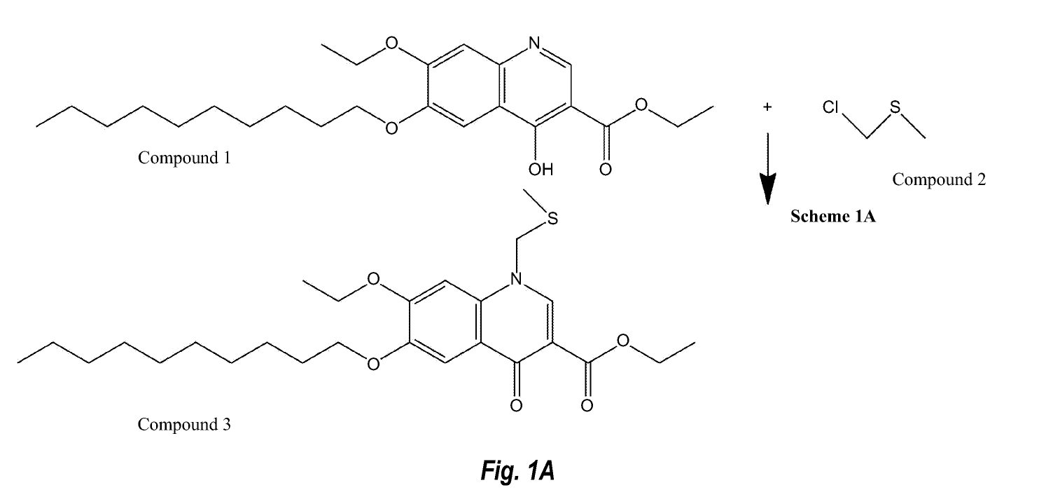

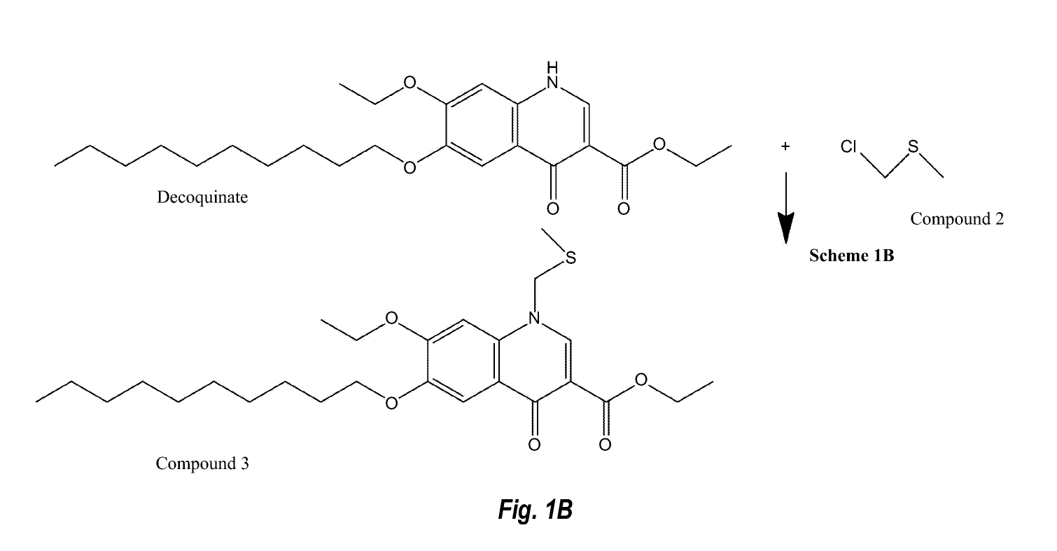

FIGS. 1A and 1B show embodiments of a step in the synthesis of a decoquinate prodrug, which step is Scheme 1A or Scheme 1B. As shown in Scheme 1A, about 0.105 g (0.25 mmol) of Compound 1 (e.g., decoquinate derivative) is combed with about 0.04 g (0.41 mmol) of Compound 2 (e.g., chloromethyl(methyl)sulfide) in DMF (e.g., 3 mL) and NaOH (e.g., 0.040 g or 0.60 mmol.) at 0° C. for ½ hour and then at room temperature overnight. Then, the solvent is removed, and the product is dissolved in EtOAc, washed with water, the water is removed, and purified on a separation column (e.g., column separation with Ethyl Acetate) to get about a 50% yield of Compound 3 (e.g., nitrogen activated decoquinate or decoquinate methyl sulfide), which is about 0.06 g (0.126 mmol.). The reaction was confirmed with TLC.

As shown in Scheme 1B, decoquinate is reacted with 1.2 equivalents of Compound 2 (e.g., chloromethyl(methyl)sulfide) in DMF in the presence of NaH (e.g., sodium hydride) at 60° C. for 4 hours. The product is isolated by column chromatography on silica gel (20:1 CHCl₃/MeOH).

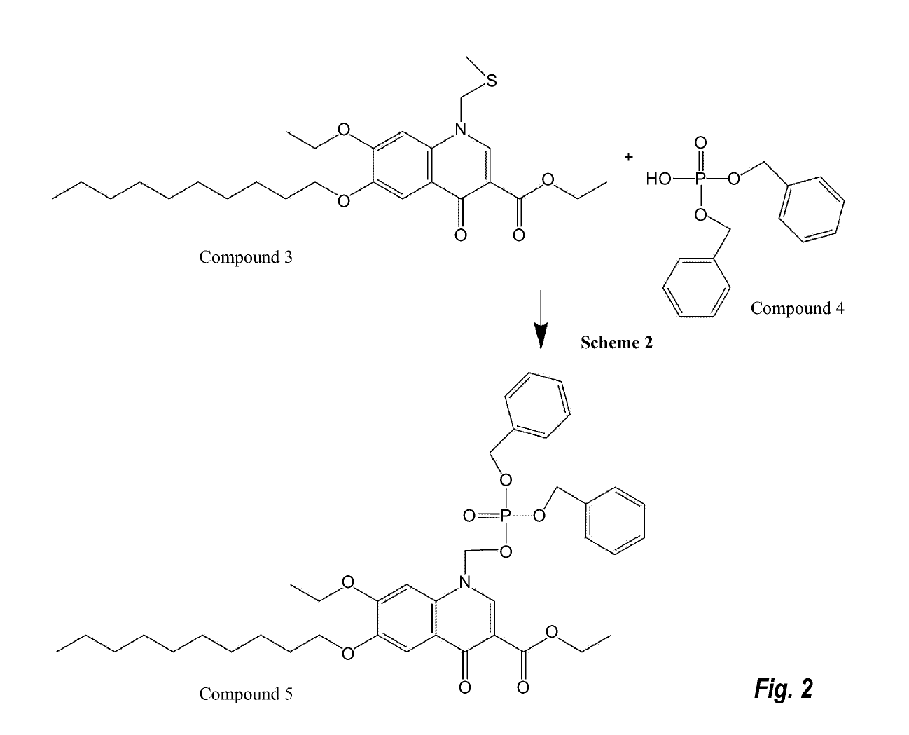

FIG. 2 shows an embodiment of a step in the synthesis of a decoquinate prodrug, which step is Scheme 2. As shown, about 0.06 g (0.126 mmol.) of Compound 3 is combined with about 0.110 g (0.395 mmol.) of Compound 4 (e.g., dibenzyl phosphate) with N-iodo-succinimide (e.g., NIS at 0.110 g or 0.49 mmol.) in THF (e.g., 2 mL) and CH₂Cl₂ (e.g., 2 mL) at room temperature for 1 hour. Then the solvent is removed and purified on a separation column (e.g., EAH to 10% MeOH:CHCl₃) to get about a 100% yield of Compound 5 (e.g., decoquinate prodrug or prodrug precursor with benzyl protecting group), which is about 0.89 g.

Also, Scheme 2 can be conducted by dissolving Compound 3 (e.g., decoquinate methyl sulfide) in CH₂Cl₂ and reacted with Compound 4 (e.g., dibenzyl phosphate) (1.2 eq.) in the presence of NIS (1.2 eq.) and powdered, activated molecular sieves for 3 hours at room temperature. Completion of the reaction is monitored by mass spectroscopy, and the product Compound 5 is isolated by column chromatography (e.g., SiO₂, EtOAc). In this reaction, the iodonium ion is generated which attaches to the sulfide making it into a good leaving group, which is displaced by the dibenzyl phosphate (e.g., Compound 4).

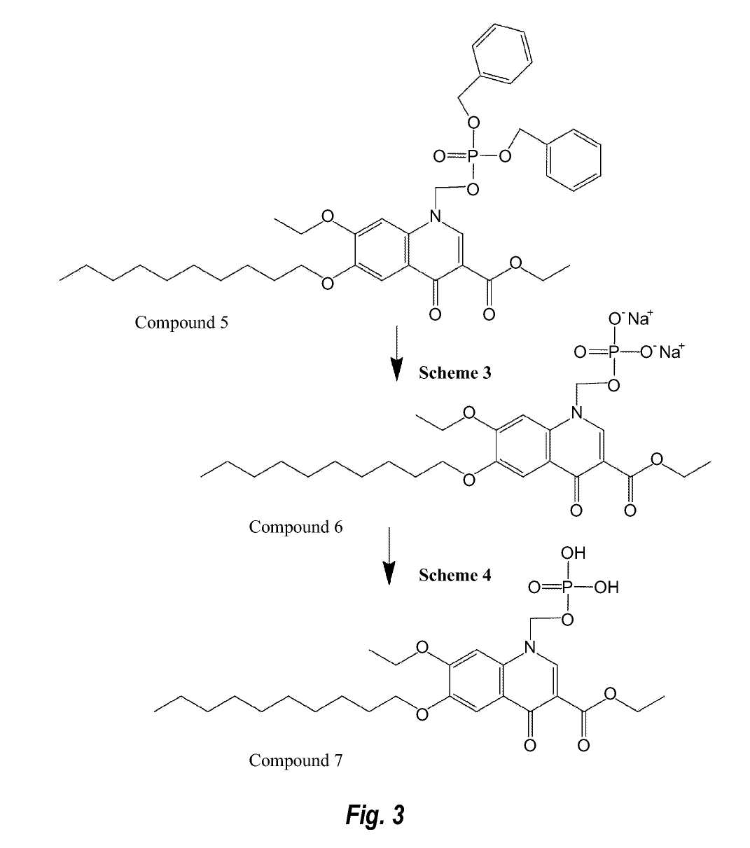

FIG. 3 shows an embodiment of a step in the synthesis of a decoquinate prodrug, which step is Scheme 3. As shown, about 0.13 g (0.018 mmol.) of Compound 5 is reacted in ethanol (e.g., 5 mL) with Pd/C (e.g., 10 mg) and with H₂O+Na₂CO₃ (e.g., 2.2 mg/0.5 mL water) under hydrogen gas (e.g., H₂) at room temperature for 1 hour. Then, the product is filtered, the solvent is removed, and the product is lyophilized to result in about 100% yield of Compound 6 (e.g., decoquinate prodrug or prodrug salt), which is about 10.45 mg.

Also, the dibenzyl phosphate of decoquinate can be subjected to hydrogenolysis to remove the benzyl groups from the phosphate to generate disodium salt of the decoquinate-POM prodrug (e.g., Compound 6). The hydrogenation can be performed at atmospheric pressure. Sodium carbonate (1 eq.) as a solution in water can be added to in situ convert the free acid phosphates to the sodium salts. After hydrogenation is complete, the catalyst is removed by filtration and the decoquinate-POM prodrug (e.g., Compound 6) is isolate as a white, fluffy powder by lyophilization of the mixture.

FIG. 3 also shows an embodiment of another step in the synthesis of a decoquinate prodrug, which step is Scheme 4. Here, Compound 6 is desalted so as to form Compound 7 (e.g., (e.g., decoquinate prodrug of Formula 5).

The synthetic protocol including Schemes 1A or 1B to Scheme 4 provides an example of a method of synthesizing the decoquinate prodrugs as described herein. Based on the chemical structures shown in the different formulae, modifications to the reaction schemes can be made to prepare any of the decoquinate prodrugs described herein.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

Additional information regarding the prodrug entities, preparation thereof, and return to drug can be found in U.S. Pat. No. 5,985,856 and U.S. application Ser. No. 13/310,087 and Dhareshwar et al. “A Novel Prodrug Strategy For Beta-Dicarbonyl Carbon Acids: Syntheses and Evaluation of the Physiochemical Characteristics of C-Phosphoryloxymethyl (POM) and Phosphoryloxymethyloxymethyl (POMOM) Prodrug Derivatives, Journal of Pharmaceutical Sciences; Vol. 99, No. 6: 2711-23 (2010), which are incorporated herein by specific reference in their entirety.

1. A compound comprising:
a structure having decoquinate coupled to a prodrug moiety, or derivative or isomer or pharmaceutically acceptable salt thereof.

2. The compound of claim 1, wherein the prodrug moiety is coupled to the nitrogen of the decoquinate or derivative or isomer or pharmaceutically acceptable salt thereof.

3. The compound of claim 2, wherein the prodrug moiety is linked to the decoquinate or derivative or isomer or pharmaceutically acceptable salt thereof through a linker.

4. The compound of claim 3, wherein the linker includes a straight aliphatic, branched aliphatic, cyclic aliphatic, heterocyclic aliphatic, substituted aliphatic, unsubstituted aliphatic, saturated aliphatic, unsaturated aliphatic, aromatic, polyaromatic, substituted aromatic, hetero-aromatic, amine, primary amine, secondary amine, tertiary amine, aliphatic amine, carbonyl, carboxyl, amide, ester, amino acid, peptide, polypeptide, sugars, sugar mimic, derivatives thereof, or combinations thereof.

5. The compound of claim 2, wherein structure is one of Formula 1 or Formula 1A or Formula 1B or Formula 1C or Formula 1D or Formula 1E or decoquinate derivative or isomer or pharmaceutically acceptable salt thereof: 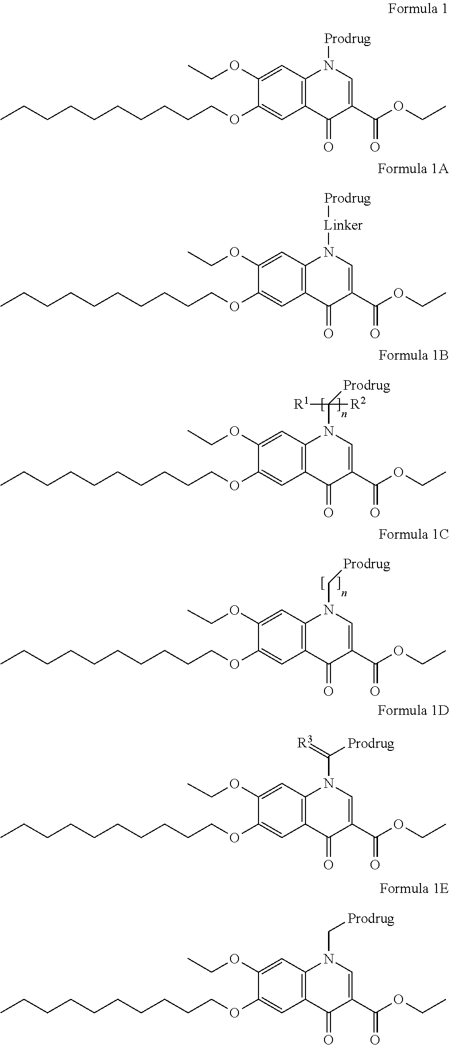
wherein:
Prodrug is a prodrug moiety;
R¹ and R² are independently selected from or include hydrogen, halogens, hydroxyls, alkoxys, straight aliphatics, branched aliphatics, cyclic aliphatics, heterocyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, sugars, sugar mimics, derivatives thereof, wherein the aliphatic groups can include carbon chains, each independently being about 1-20, about 1-10, which carbon chains may be substituted with hetero atoms O, N, S, or P, or R¹ and R² form a ring that is aliphatic or aromatic with 3, 4, 5, 6, or 7 carbon and/or hetero atom members in the ring;
R³, R⁴, and R⁵ are independently selected from or include hydrogen, halogens, hydroxyls, alkoxys, straight aliphatics, branched aliphatics, cyclic aliphatics, heterocyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, sugars, sugar mimics, derivatives thereof, wherein the aliphatic groups can include carbon chains, each independently being about 1-20, about 1-10, which carbon chains may be substituted with hetero atoms O, N, S, or P;
the linker includes a straight aliphatic, branched aliphatic, cyclic aliphatic, heterocyclic aliphatic, substituted aliphatic, unsubstituted aliphatic, saturated aliphatic, unsaturated aliphatic, aromatic, polyaromatic, substituted aromatic, hetero-aromatic, amine, primary amine, secondary amine, tertiary amine, aliphatic amine, carbonyl, carboxyl, amide, ester, amino acid, peptide, polypeptide, sugars, sugar mimic, derivatives thereof, or combinations thereof; and
n is an integer.

6. The compound of claim 5, wherein structure is one of Formula 2 or Formula 2A or Formula 2B or Formula 2C or Formula 2D or Formula 2E or decoquinate derivative or isomer or pharmaceutically acceptable salt thereof: 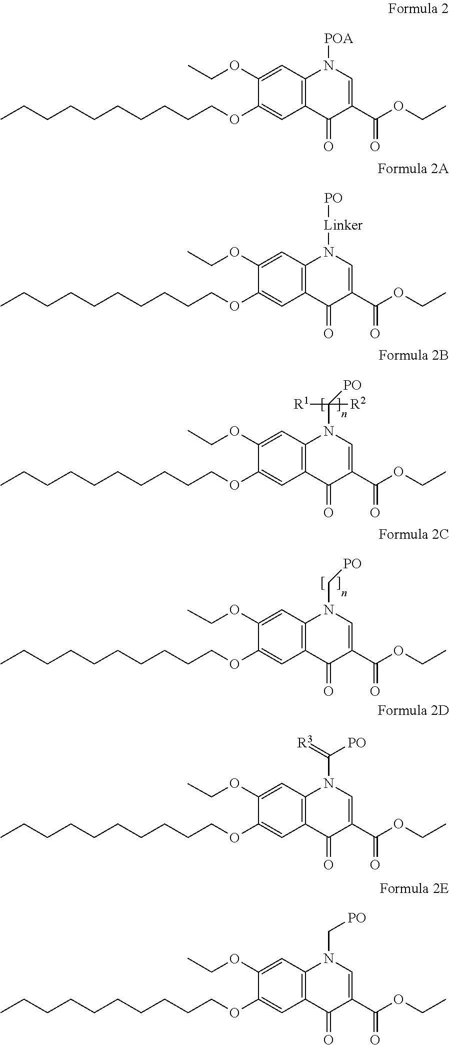
wherein:
PO is one of PO¹ or PO²; and
X is a cation

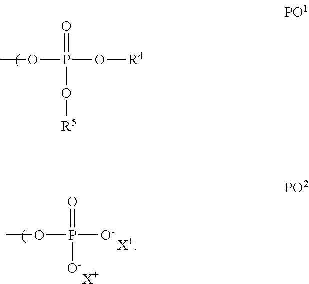

7. The compound of claim 5, wherein structure is one of Formula 3 or Formula 3A or Formula 3B or Formula 3C or Formula 3D or Formula 3E or decoquinate derivative or isomer or pharmaceutically acceptable salt thereof: 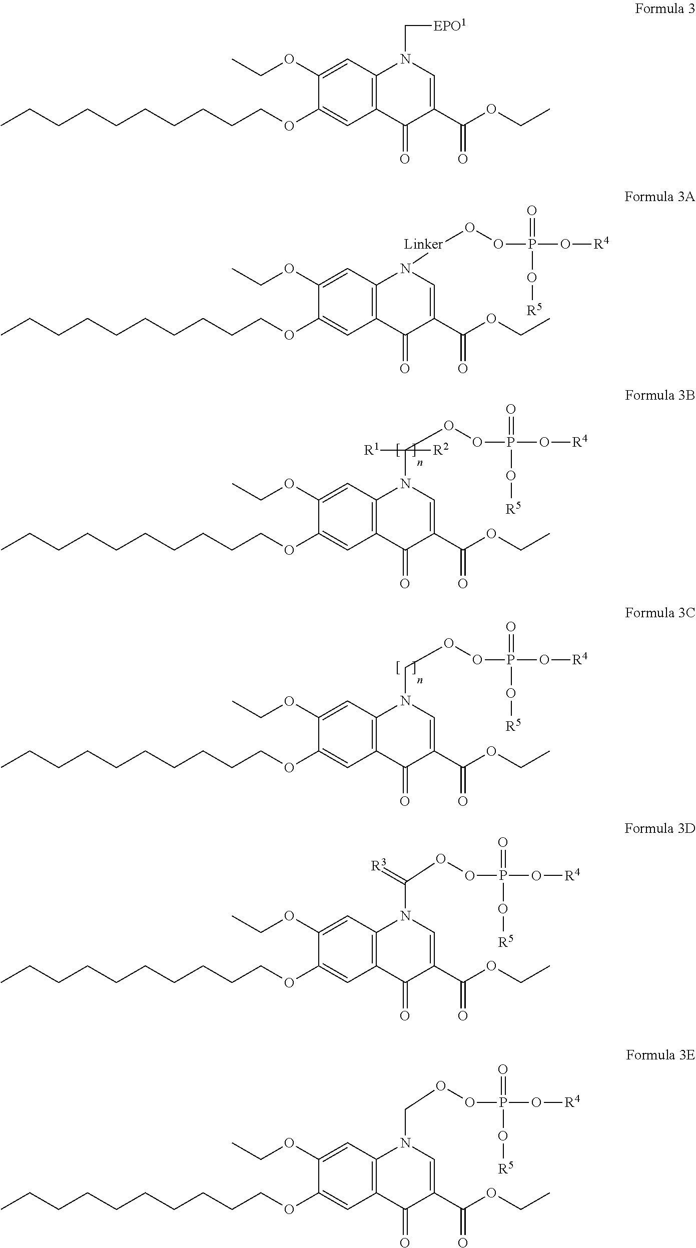
wherein EPO¹ is:

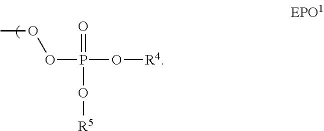

8. The compound of claim 5, wherein structure is one of Formula 4 or Formula 4A or Formula 4B or Formula 4C or Formula 4D or Formula 4E or decoquinate derivative or isomer or pharmaceutically acceptable salt thereof: 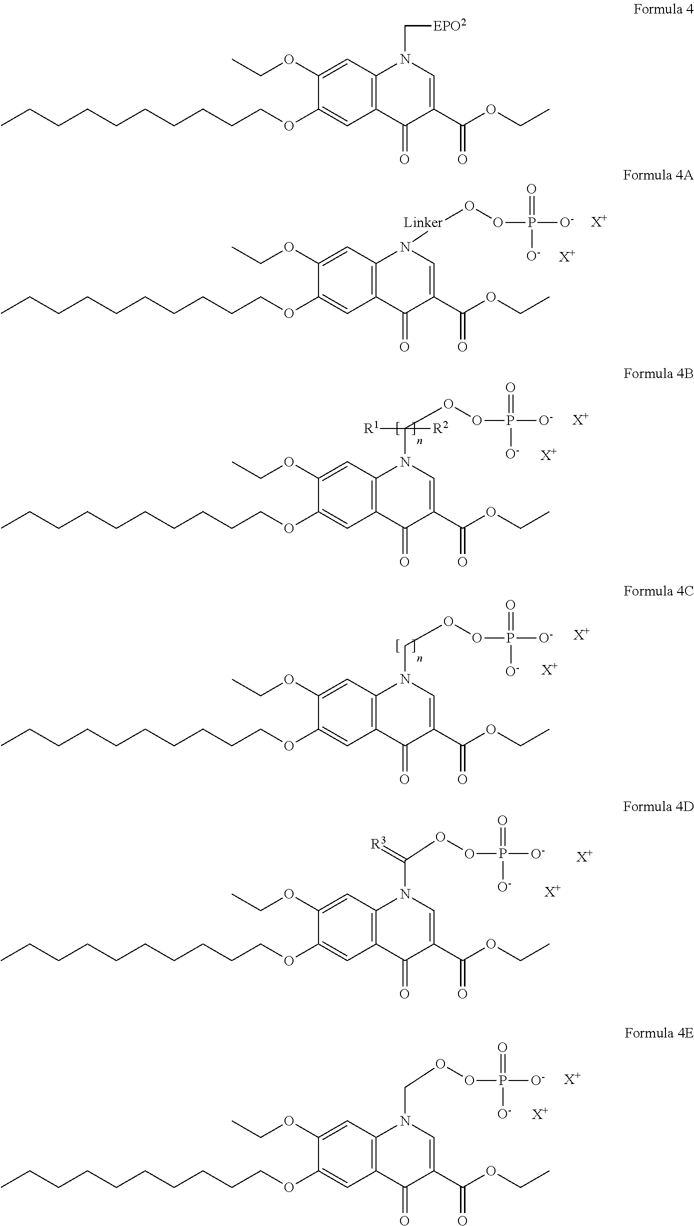
wherein:
X is a cation; and
EPO² is:

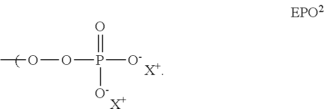

9. The compound of claim 5, wherein structure is Formula 5 or decoquinate derivative or isomer or pharmaceutically acceptable salt thereof:

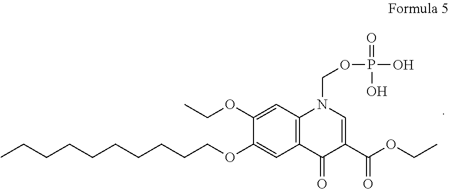 [SMILES: CCCCCCCCCCOc1cc2c(=O)c(C(=O)OCC)cn(COP(=O)(O)O)c2cc1OCC]

10. The compound of claim 5, wherein structure is Formula 6 or decoquinate derivative or isomer or pharmaceutically acceptable salt thereof:

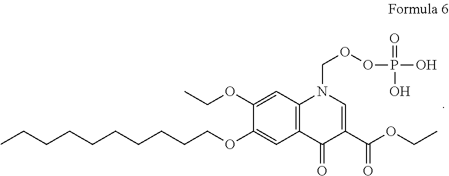 [SMILES: CCCCCCCCCCOc1cc2c(=O)c(C(=O)OCC)cn(COOP(=O)(O)O)c2cc1OCC]

11. A pharmaceutical composition comprising:
a prodrug structure having decoquinate coupled to a prodrug moiety, or derivative or isomer or pharmaceutically acceptable salt thereof; and
a pharmaceutically acceptable carrier.

12. The pharmaceutical composition of claim 11, wherein the pharmaceutically acceptable carrier includes an aqueous composition.

13. The pharmaceutical composition of claim 11, further comprising another anti-malarial agent.

14. The pharmaceutical composition of claim 11,
wherein prodrug structure is one of Formula 1 or Formula 1A or Formula 1B or Formula 1C or Formula 1D or Formula 1E or decoquinate derivative or isomer or pharmaceutically acceptable salt thereof: 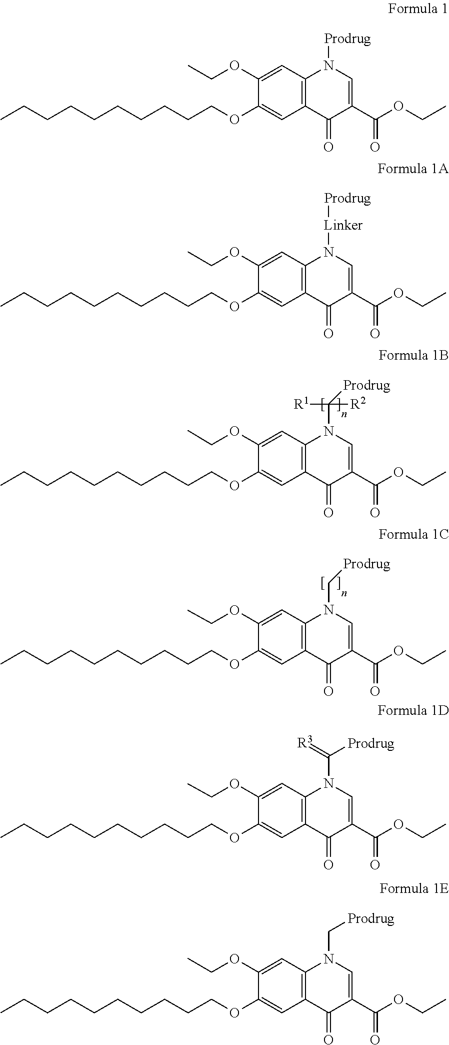
wherein:
Prodrug is a prodrug moiety;
R1 and R2 are independently selected from or include hydrogen, halogens, hydroxyls, alkoxys, straight aliphatics, branched aliphatics, cyclic aliphatics, heterocyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, sugars, sugar mimics, derivatives thereof, wherein the aliphatic groups can include carbon chains, each independently being about 1-20, about 1-10, which carbon chains may be substituted with hetero atoms O, N, S, or P, or R1 and R2 form a ring that is aliphatic or aromatic with 3, 4, 5, 6, or 7 carbon and/or hetero atom members in the ring;
R3, R4, and R5 are independently selected from or include hydrogen, halogens, hydroxyls, alkoxys, straight aliphatics, branched aliphatics, cyclic aliphatics, heterocyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, sugars, sugar mimics, derivatives thereof, wherein the aliphatic groups can include carbon chains, each independently being about 1-20, about 1-10, which carbon chains may be substituted with hetero atoms O, N, S, or P;
the linker includes a straight aliphatic, branched aliphatic, cyclic aliphatic, heterocyclic aliphatic, substituted aliphatic, unsubstituted aliphatic, saturated aliphatic, unsaturated aliphatic, aromatic, polyaromatic, substituted aromatic, hetero-aromatic, amine, primary amine, secondary amine, tertiary amine, aliphatic amine, carbonyl, carboxyl, amide, ester, amino acid, peptide, polypeptide, sugars, sugar mimic, derivatives thereof, or combinations thereof; and
n is an integer.

15. The pharmaceutical composition of claim 14, wherein structure is one of Formula 2 or Formula 2A or Formula 2B or Formula 2C or Formula 2D or Formula 2E or decoquinate derivative or isomer or pharmaceutically acceptable salt thereof: 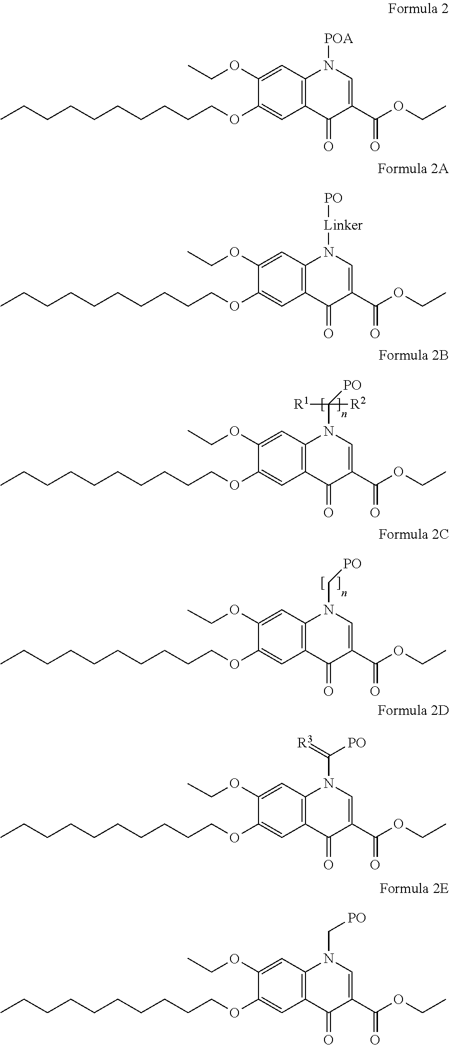
wherein:
PO is one of PO¹ or PO²; and
X is a cation

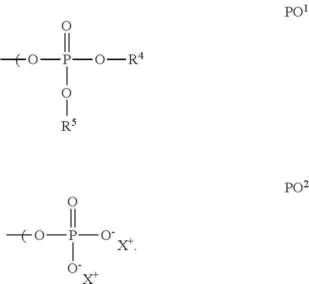

16. The pharmaceutical composition of claim 14, wherein structure is one of Formula 3 or Formula 3A or Formula 3B or Formula 3C or Formula 3D or Formula 3E or decoquinate derivative or isomer or pharmaceutically acceptable salt thereof: 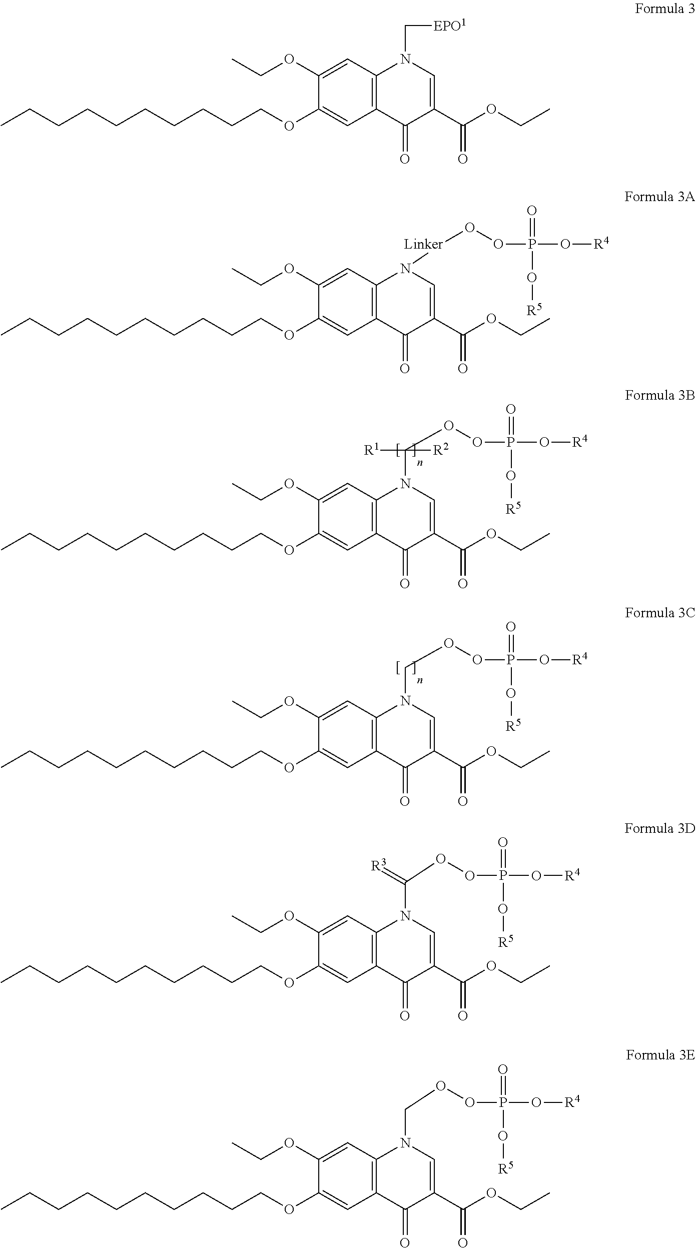
wherein EPO¹ is:

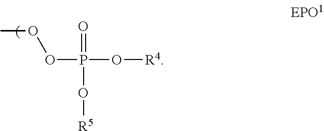

17. The pharmaceutical composition of claim 14, wherein structure is one of Formula 4 or Formula 4A or Formula 4B or Formula 4C or Formula 4D or Formula 4E or decoquinate derivative or isomer or pharmaceutically acceptable salt thereof: 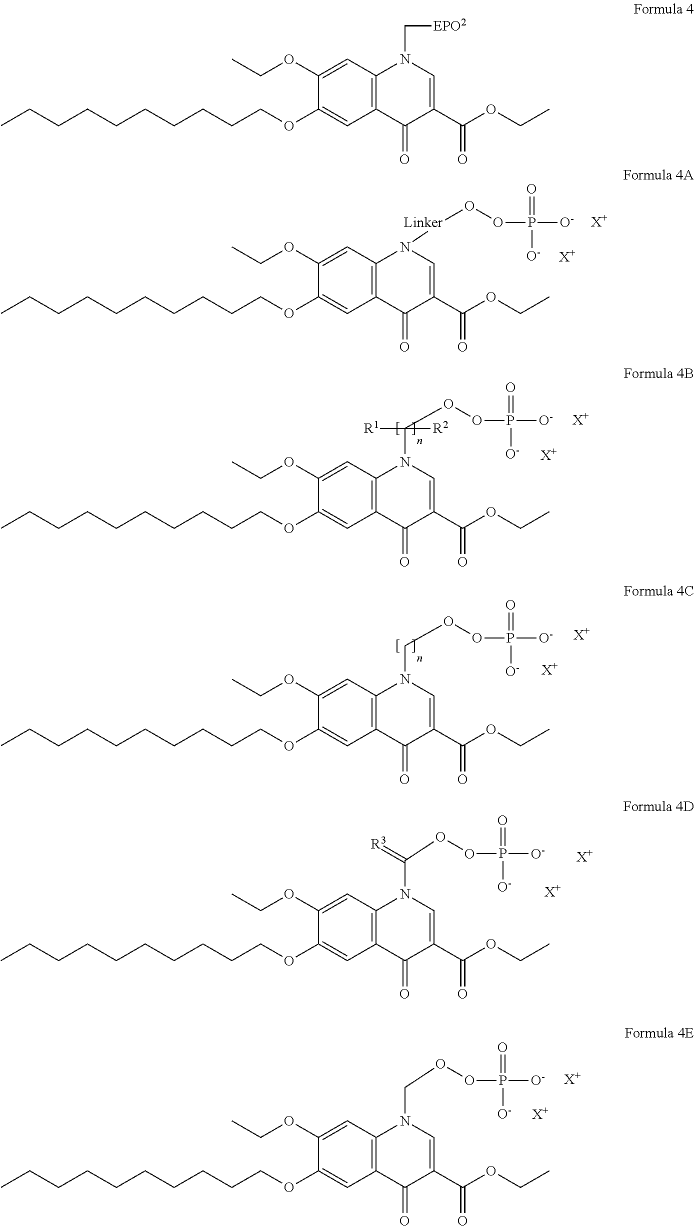
wherein:
X is a cation; and
EPO² is:

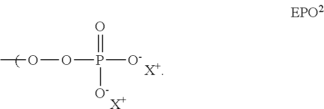

18. The pharmaceutical composition of claim 14, wherein structure is Formula 5 or decoquinate derivative or isomer or pharmaceutically acceptable salt thereof:

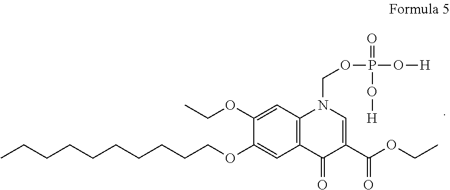 [SMILES: CCCCCCCCCCOc1cc2c(=O)c(C(=O)OCC)cn(COP(=O)(O)O)c2cc1OCC]

19. The pharmaceutical composition of claim 14, wherein structure is Formula 6 or decoquinate derivative or isomer or pharmaceutically acceptable salt thereof:

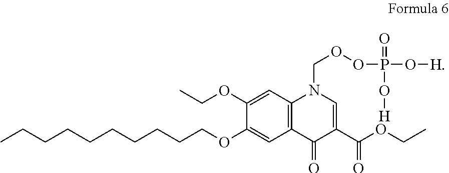

20. A method of inhibiting or treating a parasitic infection, the method comprising:
providing a subject in need of inhibition or treatment for a parasitic infection; and
administering to the subject a pharmaceutical composition containing a therapeutically effective amount of a decoquinate prodrug.

21. The method of claim 20, wherein the parasitic infection is malaria.

22. The method of claim 20, wherein the parasitic infection is a coccidian infection.
